(19) United States
(12) Patent Application Publication
(10) Pub. No.: US 2011/0054589 A1
(43) Pub. Date: Mar. 3, 2011

(54) STENT WITH VARIABLE CROSS SECTION BRAIDING FILAMENT AND METHOD FOR MAKING SAME

(75) Inventors: Mehran Bashiri, San Carlos, CA (US); Leila Bahreinian, Santa Clara, CA (US)

(73) Assignee: Boston Scientific Scimed, Inc., Maple Grove, MN (US)

(21) Appl. No.: 12/869,474

(22) Filed: Aug. 26, 2010

Related U.S. Application Data
(60) Provisional application No. 61/237,431, filed on Aug. 27, 2009.

(51) Int. Cl. A61F 2/82; D04C 1/00

(52) U.S. Cl. 623/1.15; 87/9

(57) ABSTRACT

A braided stent comprises a filament having at least one circular zone and at least two non-circular zones. Embodiments of the braided stent have a proximal segment, a middle segment, and a distal segment, wherein a porosity of the middle segment is lower than, a respective porosity of the proximal and distal segments. In one embodiment, a radial pressure of the middle segment is separately controlled to be different from, e.g., less than, a radial pressure of the distal segment. In another embodiment, a stiffness of the middle segment is separately controlled to be different from, e.g., less than, a stiffness of the distal segment.

### RELATED APPLICATION DATA

This application claims the benefit under 35 U.S.C. §119 to provisional application Ser. No. 61/237,431, filed Aug. 27, 2009, which is incorporated by reference into the present application in its entirety.

### FIELD OF THE INVENTION

The field of the invention generally relates to devices, such as stents, for reinforcing the structural integrity of vessels of a human or veterinary patient. More particularly, the field of the invention relates to stents with variable porosity.

### BACKGROUND OF THE INVENTION

Stents, grafts, stent-grafts, vena cava filters and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, etc. Stents may be used to reinforce body vessels and to prevent restenosis following angioplasty in the vascular system. They may be self-expanding, mechanically expandable or hybrid expandable.

Stents are generally tubular devices for insertion into body lumens. However, it should be noted that stents may be provided in a wide variety of sizes and shapes. Balloon expandable stents require mounting over a balloon, positioning, and inflation of the balloon to expand the stent radially outward. Self-expanding stents expand into place when unconstrained, without requiring assistance from a balloon. A self-expanding stent may be biased so as to expand upon release from the delivery catheter and/or include a shape-memory component which allows the stent to expand upon exposure to a predetermined condition. Some stents may be characterized as hybrid stents which have some characteristics of both self-expandable and balloon expandable stents.

Due to the branching nature of the human vasculature it is not uncommon for stenoses to form at any of a wide variety of vessel bifurcations. A bifurcation is an area of the vasculature or other portion of the body where a first (or parent) vessel is bifurcated into two or more branch vessels. In some cases it may be necessary to implant multiple stents at the bifurcation in order to address a stenosis located thereon. Alternatively, a stent may be provided with multiple sections or branches that may be deployed within the branching vessels of the bifurcation.

Stents may be constructed from a variety of materials such as stainless steel, Elgiloy, nickel, titanium, nitinol, shape memory polymers, etc. Stents may also be formed in a variety of manners as well. For example a stent may be formed by etching or cutting the stent pattern from a tube or sheet of stent material; a sheet of stent material may be cut or etched according to a desired stent pattern whereupon the sheet may be rolled or otherwise formed into the desired substantially tubular, bifurcated or other shape of the stent; one or more wires or ribbons of stent material may be woven, braided or otherwise formed into a desired shape and pattern. The density of the braid in braided stents is measured in picks per inch. Stents may include components that are welded, bonded or otherwise engaged to one another.

Typically, a stent is implanted in a blood vessel or other body lumen at the site of a stenosis or aneurysm by so-called “minimally invasive techniques” in which the stent is compressed radially inwards and is delivered by a catheter to the site where it is required through the patient's skin or by a “cut down” technique in which the blood vessel concerned is exposed by minor surgical means. When the stent is positioned at the correct location, the stent is caused or allowed to expand to a predetermined diameter in the vessel.

Flow diverting stents may treat a brain aneurysm by providing resistance to blood in-flow to the aneurysm. Subsequently, the blood in the aneurysm stagnates and, in time, forms a thrombosis to close the aneurysm. To increase the therapeutic effectiveness of a flow diverting stent, the middle segment of the stent, which impedes blood flow into the aneurysm, has a low porosity.

Porosity of stent material is a measure of the tendency of that material to allow passage of a fluid. A stent material's porosity index (PI) is defined as one minus the ratio of stent metal surface area to artery surface area covered by the stent. Higher porosity means that the stent material has less metal surface area compared to artery surface area and lower porosity means that the stent has more metal surface area compared to artery surface area.

FIG. 13 shows a stent that has been cut open along its length and unrolled into a flat sheet. The proximal to distal longitudinal axis stretches from left to right. The braid angle of a stent between two braid filaments is labeled as alpha. There are three states in which a stent's braid angle is measured: (1) when the stent is fully expanded with no restriction; (2) when the stent is compressed to fit into a catheter; and (3) when the stent is expanded in a vessel. Flaring the ends of a stent can add a fourth state.

The number of wires in a stent determines the type of braiding apparatus, i.e. 32 wires vs. 48 wires. Wire diameter also affects porosity, radial pressure, and stiffness of a stent.

Perceived problems with current stents include increasing radial stiffness with decreasing porosity by increasing picks per inch. The increased radial stiffness results in resistance to radial compression, which is needed to collapse the stent for insertion through an intravascular catheter. Stents have been braided with ribbons instead of wire with a circular cross section to decrease porosity without an undue increase in radial stiffness, but such stents have unacceptably low radial pressure at the anchoring ends. Further, such stents do not form desirable looped end designs well, because it is challenging to maintain the ribbon in a single plane while forming a loop. Another perceived problem with current stents is that braiding stents from either ribbon or wire with a circular cross section results in limited porosity gradient between ends, where high porosity is desirable, and the middle, where low porosity is desirable.

### SUMMARY

In accordance with a general aspect of the inventions disclosed herein, a braided stent is formed from a filament having at least one circular zone and at least two non-circular zones. Embodiments of the braided stent may have a proximal segment, a middle segment, and a distal segment. In one such embodiment, a porosity of the middle segment is lower than a respective porosity of the proximal and distal segments. In another such embodiment, a radial pressure of the middle segment may be controlled separately from, e.g., so that it is less than, a radial pressure of the distal segment. By way of another example, a stiffness of the middle segment may also be controlled separately from, e.g., so that it is less than, a stiffness of the distal segment.

In one embodiment, the filament comprising a single circular zone and two non-circular zones, wherein the circular zone is disposed between the two non-circular zones. Optionally, the circular zone may have at least one looped end. In one embodiment, the filament has three circular zones and two non-circular zones, wherein the three circular zones and the two non-circular zones are alternately disposed on the filament.

In accordance with another aspect of the disclosed inventions, a method of braiding a stent includes providing a filament having at least one circular zone and at least two non-circular zones; and braiding the filament into a stent. In one such embodiment, the method further comprises wrapping at least one circular zone of the filament around a mandrel to form a distal loop of the stent. In one such embodiment, the method further comprises braiding at least one non-circular zone of the filament into a low porosity stent segment. In one such embodiment, the method further comprises braiding at least one circular zone of the filament into a high radial pressure stent segment.

In one embodiment, the filament comprises a single circular zone and two non-circular zones, the method further comprising braiding the circular zone into a high porosity distal stent segment, braiding respective medial portions of the two non-circular zones into a low porosity middle stent segment, and braiding respective lateral portions of the two non-circular zones into a high porosity proximal stent segment.

Other and further aspects and embodiments will become apparent from the figures and following detailed description thereof.

### BRIEF DESCRIPTION OF THE DRAWINGS

Referring now to the drawings in which like reference numbers represent corresponding parts throughout, and in which:

FIG. 1 is a perspective view of a stent filament in accordance with one embodiment of the invention.

FIGS. 2A, 2B, and 2C are cross-sectional views through the lines 2A-2A, 2B-2B, and 2C-2C in FIG. 1, respectively.

FIG. 3 is a perspective view of a stent in accordance with one embodiment of the invention.

FIGS. 4A, 4B, and 4C are cross-sectional views through the filament zones in the proximal, middle, and distal segments of the stent in FIG. 3, respectively.

FIG. 5 is a perspective view of a stent filament in accordance with another embodiment of the invention.

FIGS. 6A, 6B, 6C, 6D, and 6E are cross-sectional views through the lines 6A-6A, 6B-6B, 6C-6, 6D-6D, and 6E-6E in FIG. 5, respectively.

FIG. 7 is a perspective view of a stent in accordance with another embodiment of the invention.

FIGS. 8A, 8B, and 8C are cross-sectional views through the filament zones in the proximal, middle, and distal segments of the stent in FIG. 7, respectively.

FIG. 9 is a perspective view of a stent filament and a mandrel used to braid a stent in accordance with one embodiment of the invention, where the portion of the stent filament behind the mandrel is shown in shadow for clarity.

FIGS. 10-12 are detailed perspective views of braids in accordance with various embodiments of the invention.

FIG. 13 shows (for purposes of illustration) a stent that has been cut open along its length and unrolled into a flat sheet.

### DETAILED DESCRIPTION OF THE ILLUSTRATED EMBODIMENTS

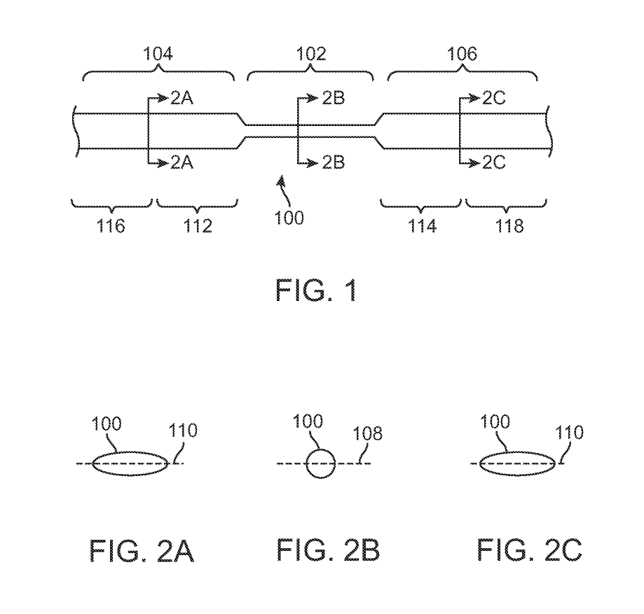

FIG. 1 illustrates a stent filament 100 according to an embodiment of the invention. The filament 100 may be formed from both metallic and non-metallic materials.

Metallic filament materials include, without limitation, nitinol, stainless steel, cobalt-based alloy such as Elgiloy, platinum, gold, titanium, tantalum, niobium, and combinations thereof and other biocompatible materials, as well as polymeric materials. The filament 100 or zones thereof may have an inner core of tantalum, gold, platinum, iridium or combinations thereof and an outer member or layer of nitinol to provide a composite filament for improved radiopacity or visibility. Non-metallic materials include, without limitation, polyesters, such as polyethylene terephthalate (PET) polyesters, polypropylenes, polyethylenes, polyurethanes, polyolefins, polyvinyls, polymethylacetates, polyamides, naphthalane dicarboxylene derivatives, natural silk, and polytetrafluoroethylenes. Non-metallic materials also include carbon, glass, and ceramics. Stents braided from filament 100 made from memory material, e.g. nitinol, could be biased to take on an expanded form due to the memory property of the filament material. The expanded form of the stent could be a generally tubular shape with flared ends. The flared ends increase radial pressure and stent stiffness for better anchoring at the ends of the stent, especially the distal end.

The filament 100 has three zones, one circular zone 102 and two non-circular zones 104, 106. The cross section of the filament 100 in the circular zone 102 is circular, as shown in FIG. 2B. The cross section of the filament 100 in the non-circular zones 104, 106 is non-circular, including rectangular, concave, and ovoid, as shown in FIGS. 2A and 2C. The filament 100 in the circular zone 102 may be shaped like a wire and the filament 100 in the non-circular zone 102 may be shaped like a ribbon. The cross sectional shapes of the various filament zones 102, 104, and 106 may be configured either during or after formation of the filament 100.

The filament 100 in the non-circular zones 104, 106 has a lower moment of area in the flat direction, making it more flexible than filament 100 in the circular zone 102. Increasing flexibility reduces the radial pressure exerted by a stent segment braided from filament 100 in the non-circular zones 104, 106 compared to a stent segment braided from filament 100 in the circular zone 102 with the same braid angle and braid diameter. Also, the filament 100 in the non-circular zones 104, 106 is wider than filament 100 in the circular zone 102. For instance, the diameter 108 of the circular cross section measures 0.002 inches and the long axis 110 of the ovoid cross section measures 0.003 inches. Increasing width decreases the porosity of a stent segment braided from filament 100 in the non-circular zones 104, 106 compared to a stent segment braided from filament 100 in the circular zone 102.

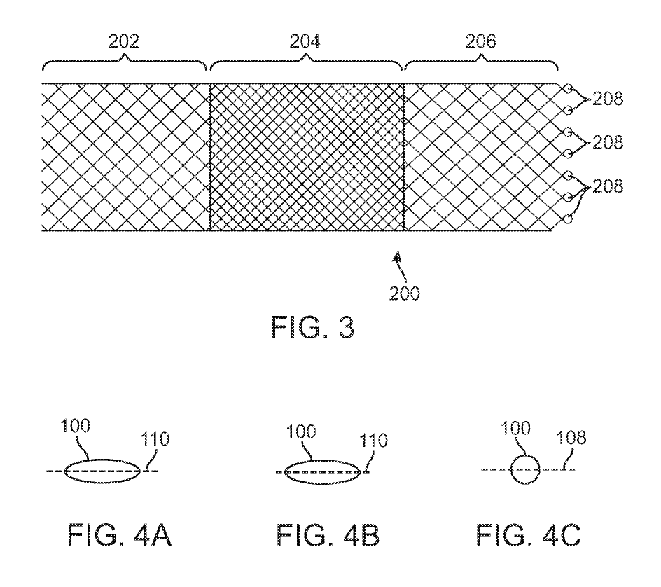

The stent 200 braided from the filament 100 is shown in FIG. 3. The stent 200 has three segments, a proximal segment 202, a middle segment 204, and a distal segment 206. The distal segment 206 ends in distal loops 208. The distal segment 206 of the stent 200 is braided from filament 100 in the circular zone 102. The middle segment 204 of the stent 200 is braided from filament 100 in the non-circular zones 104, 106. As such, the middle segment 204 of the stent 200 has lower porosity and exerts lower radial pressure compared to the distal segment 206 of the stent 200, given the same braid angle and braid diameter. The lower porosity of the middle segment 204 increases the flow diverting effectiveness of the stent 200. The higher radial pressure exerted by the distal segment 206 provides a better anchor for the stent 200.

The non-circular shaped cross section of the filament 100 in the non-circular zones 104, 106 also reduces the stiffness, both radial and axial, of the middle segment 204 of the stent 200, which is braided from filament 100 in the non-circular zones 104, 106. The reduced radial pressure and stiffness allow the middle segment 204 of the stent 200 to be braided more densely, i.e., higher picks per inch, while maintaining a radial pressure and a stiffness respectively less than or equal to the radial pressure and stiffness of the distal segment 206 of the stent 200, which has fewer picks per inch. This allows the middle segment 204 of the stent 200 to have higher braid density, and therefore lower porosity, than the other segments of the stent 200, while maintaining the ability to radially collapse the stent for insertion through a catheter and reducing radial stiffness.

Like the middle segment 204, the proximal segment 202 of the stent 200 is also braided from the non-circular zones 104, 106 of the filament 100. The middle segment 204 is braided from the medial portions 112, 114 of the non-circular zones 104, 106 of the filament 100. The proximal segment 202 is braided from the lateral portions 116, 118 of the non-circular zones 104, 106 of the filament 100. Unlike the middle segment 204, the braid density of the proximal segment 202 is lower due to a smaller braid angle or lower picks per inch. The resulting high porosity in the proximal segment 202 reduces the likelihood of side branch blockage.

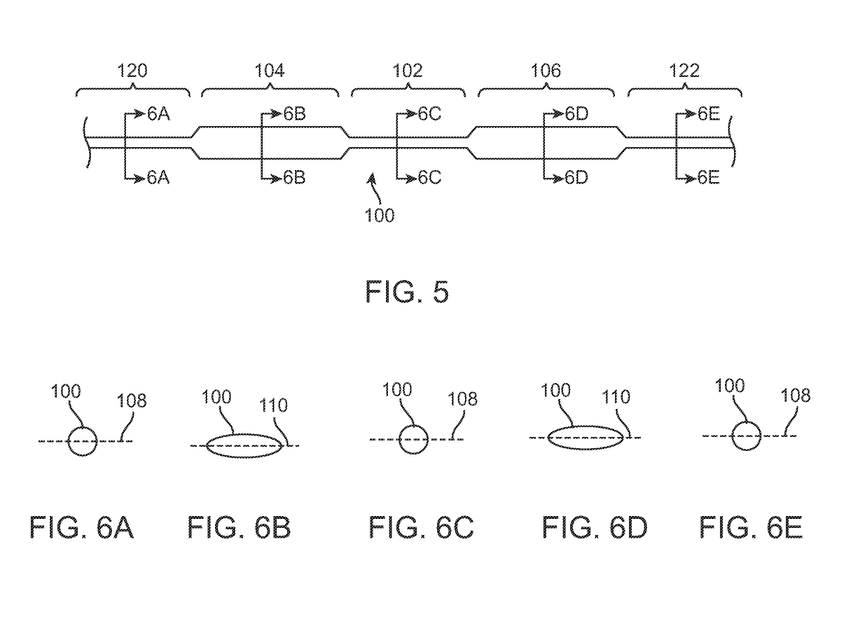

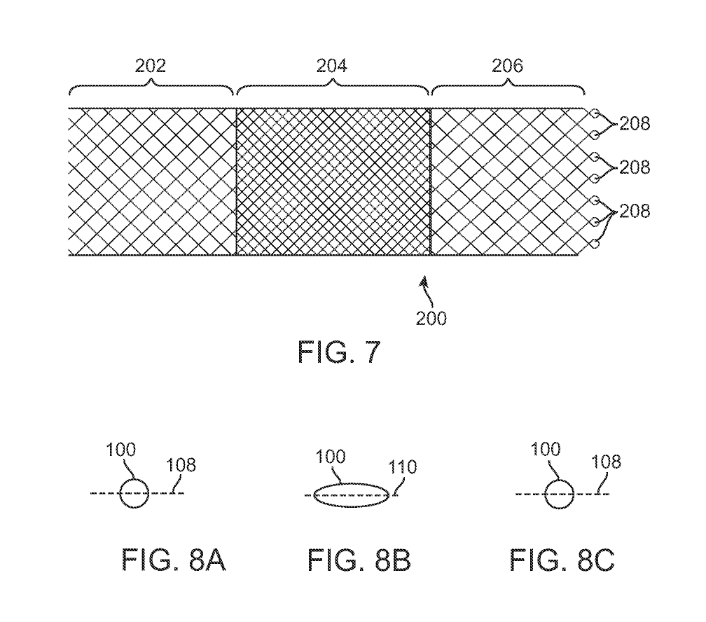

In another embodiment of the invention shown in FIGS. 5 and 6A-6E, the filament 100 has five zones, three circular zones 102, 120, 122, and two non-circular zones 104, 106. As shown in FIGS. 7 and 8A-8C, the stent 200 braided from this filament 100 is similar to the stent 200 discussed above, except that the proximal segment 202 of the stent 200 is braided from the lateral circular zones 120, 122 of the filament 100. Only the middle segment 204 of the stent 200 is braided from the non-circular zones 104, 106 of the filament 100.

As shown in FIG. 7, the proximal segment 202 of the stent 200 is identical to the distal segment 206 of the stent with the exception of the distal loops 208, which are only present in the distal segment 206. Both the proximal segment 202 and distal segment 206 of the stent 200 are braided from circular filament zones 102, 120, 122, as shown in FIGS. 8A and 8C. The middle segment 204 of the stent 200 is braided from non-circular filament zone 104, 106, as shown in FIG. 8B. Further, the middle segment 204 of the stent 200 has a higher braid density (i.e., higher picks per inch or larger Alfa angle) than the proximal segment 202 and distal segment 206 of the stent 200.

Accordingly, the middle 204 segment of the stent 200 has lower porosity than the proximal segment 202 and distal segment 206 of the stent 200. Notwithstanding the higher braid density in the middle segment 204 of the stent 200, that segment of the stent 200 has a radial pressure and a stiffness respectively less than or equal to the radial pressure and stiffness of the proximal segment 202 and distal segment 206 of the stent 200. The middle segment 204 of the stent 200 is able to maintain lower radial pressure and lower stiffness due to the non-circular shape of the filament 100 at non-circular zones 104, 106 from which it is braided.

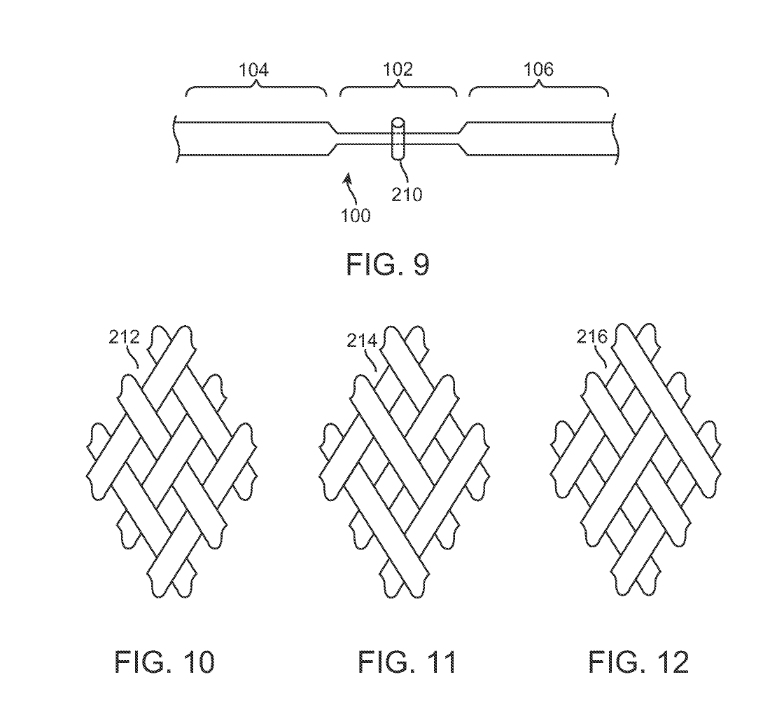

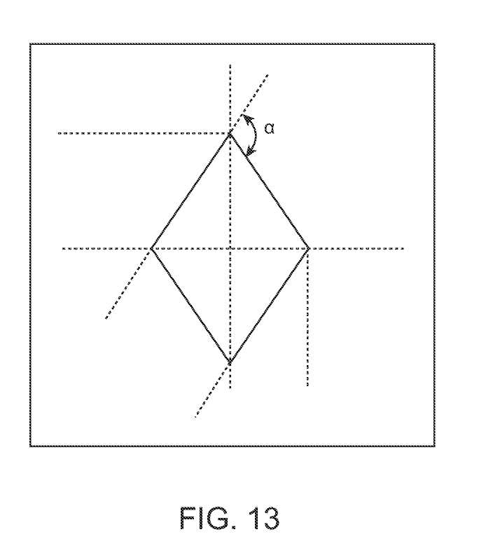

The filament 100 is braided into a stent 200 as shown in FIGS. 9-12. Braiding a filament 100 into a stent 200 begins by placing a mandrel pin 210 adjacent to the approximate middle of the middle circular zone 102 of the filament 100, as shown in FIG. 9. The filament 100 is first wrapped around the mandrel pin 210 to form a distal loop 208. The various zones of the filament 100 are then braided together to form the distal, middle, and proximal segments 206, 204, 202 of the stent 200.

As depicted in FIGS. 3 and 7, braiding of filaments 100 includes the interlacing of at least two sections of filament 100 such that the paths of the filament sections are diagonal to the stent delivery direction, forming a tubular structure. Useful braids include, but are not limited to, a diamond braid having a 1/1 intersection repeat (i.e., braid 212 as depicted in FIG. 10), a regular braid having a 2/2 intersection repeat (i.e., braid 214 as depicted in FIG. 11), and a Hercules braid having a 3/3 intersection repeat (i.e., braid 216 as depicted in FIG. 12). U.S. Pat. No. 5,653,746, the contents of which are incorporated herein by reference, further describes such braids. Moreover, a triaxial braid may also be used. A triaxial braid has at least one filament section that typically runs in the longitudinal direction or axial direction of the stent to limit filament movement. The axial or longitudinal filament section is not interlaced or interwound with the other braid filament sections, but is trapped between the different sections of filament in the braided structure. Moreover, an interlocking three-dimensional braided structure or a multi-layered braided structure is also useful. A multi-layered braided structure is defined as a structure formed by braiding wherein the structure has a plurality of distinct and discrete layers.

Generally, a braided structure is formed having a braid angle from about 30° to about 90° with respect to the longitudinal axis of the braided structure, desirably about 54.5° to about 75°. The braid angle is set by heat setting. When deploying the stent 200 into a vessel with a smaller diameter than the expanded stent 200, the angle is reduced as the stent 200 is compressed radially to fit into the vessel.

While various embodiments of the present invention have been shown and described, they are presented for purposes of illustration, and not limitation. Various modifications may be made to the illustrated and described embodiments without departing from the scope of the present invention, which is to be limited and defined only by the following claims and their equivalents.

What is claimed is:

1. A braided stent, comprising:
a filament having at least one circular zone and at least two non-circular zones, wherein the filament is braided to form the stent.

2. The braided stent of claim 1, the filament comprising a single circular zone and two non-circular zones, wherein the circular zone is disposed between the two non-circular zones.

3. The braided stent of claim 2, wherein the circular zone comprises at least one looped end.

4. The braided stent of claim 1, the stent further comprising a proximal segment, a middle segment, and a distal segment, wherein a porosity of the middle segment is lower than a respective porosity of the proximal and distal segments.

5. The braided stent of claim 4, wherein a radial pressure of the middle segment is different than a radial pressure of the distal segment.

6. The braided stent of claim 4, wherein a stiffness of the middle segment is different than a stiffness of the distal segment.

7. The braided stent of claim 1, the filament comprising three circular zones and two non-circular zones, wherein the three circular zones and the two non-circular zones are alternately disposed on the filament.

8. The braided stent of claim 7, the stent further comprising a proximal segment, a middle segment, and a distal segment, wherein a porosity of the middle segment is lower than a respective porosity of the proximal and distal segments.

9. The braided stent of claim 8, wherein a radial pressure of the middle segment is different than a respective radial pressure of each of the proximal and distal segments.

10. The braided stent of claim 8, wherein a stiffness of the middle segment is different than a respective stiffness of each of the proximal and distal segments.

11. A method of braiding a stent, comprising:
providing a filament having at least one circular zone and at least two non-circular zones; and
braiding the filament into a stent.

12. The method of claim 11, further comprising wrapping at least one circular zone of the filament around a mandrel to form a distal loop of the stent.

13. The method of claim 11, further comprising braiding at least one non-circular zone of the filament into a low porosity stent segment.

14. The method of claim 11, further comprising braiding at least one circular zone of the filament into a high radial pressure stent segment.

15. The method of claim 11, the filament comprising a single circular zone and two non-circular zones, the method further comprising
braiding the circular zone into a high porosity distal stent segment,
braiding respective medial portions of the two non-circular zones into a low porosity middle stent segment, and
braiding respective lateral portions of the two non-circular zones into a high porosity proximal stent segment.

16. The method of claim 11, the filament comprising a single circular zone and two non-circular zones, the method further comprising
braiding the circular zone into a high radial pressure distal stent segment, and
braiding respective medial portions of the two non-circular zones into a low radial pressure middle stent segment.

17. The method of claim 11, wherein the filament comprises three circular zones and two non-circular zones, the three circular zones comprising respective proximal, middle and distal circular zones, the method further comprising
braiding the middle circular zone into a high porosity distal stent segment,
braiding the two non-circular zones into a low porosity middle stent segment, and
braiding the proximal and distal circular zones into a high porosity proximal stent segment.

18. The method of claim 11, wherein the filament comprises three circular zones and two non-circular zones, the three circular zones comprising respective proximal, middle and distal circular zones, the method further comprising
braiding the middle circular zone into a high radial pressure distal stent segment,
braiding the two non-circular zones into a low radial pressure middle stent segment, and
braiding the proximal and distal circular zones into a high radial pressure proximal stent segment.
